(19) United States
(12) Patent Application Publication
(10) Pub. No.: US 2011/0105842 A1
(43) Pub. Date: May 5, 2011

(54) SUCTION DEVICE FOR LAPAROSCOPIC INSTRUMENTS AND METHOD

(76) Inventor: ROBERTO FOGEL, Miami, FL (US)

(21) Appl. No.: 12/940,811

(22) Filed: Nov. 5, 2010

Related U.S. Application Data
(60) Provisional application No. 61/258,564, filed on Nov. 5, 2009.

(51) Int. Cl. A61B 1/015; A61B 1/313; A61B 1/317

(52) U.S. Cl. 600/104; 600/158

(57) ABSTRACT

The present invention is related to a suction device especially designed for laparoscopic and/or arthroscopic instruments, including at least two cylindrical suction ports located at both sides of the distal end of the laparoscopic and/or arthroscopic instrument. Each suction port has inside and next to the distal end thereof at least one sharp traversing blade. Each suction port is connected to a suction line in turn connected to a suction pump. The external end of said suction line is connected to a disposal recipient into which the debris, blood, coagula etc removed from the surgical site will be disposed.

### BACKGROUND OF THE INVENTION

1. Field of the Invention

The present invention relates generally to laparoscopic instruments, including but not limited to mini lab instruments, pediatric length instruments, bariatric length instruments, etc., and more particularly is referred to a suction device applicable to laparoscopic instruments through which blood, coagula, debris and any other element the surgeon may find during a laparoscopic procedure.

Even more particularly, the present invention is directed to a suction device for laparoscopic instruments for removing from the surgical field blood and any other element that is obstructing the surgeon vision during the procedure.

2. Description of the Prior Art

Laparoscopic surgery, also called minimally invasive surgery (MIS), is a modern surgical technique in which a surgical procedure in different parts of the body, for example, the abdominal cavity, is performed using small incisions as access ports. These incisions are usually between 0.5 and 1.5 cm long and they represent an important advantage respect to regular incisions needed in traditional surgical procedures.

Smaller incisions are always related to lower possibilities of surgical problems for the patient. Just as an example, fewer problems with surgical wounds, fewer infections, and faster recoveries are just some of the benefits of this type of surgeries.

Laparoscopic surgery includes operations within the abdominal or pelvic cavities, whereas keyhole surgery performed on the thoracic or chest cavity is called thoracoscopic surgery. Laparoscopic and thoracoscopic surgery belong to the broader field of endoscopy.

The benefits of minimally invasive or laparoscopic procedures may be summarized as:
less post operative discomfort since the incisions are much smaller
quicker recovery times
shorter hospital stays
earlier return to full activities
much smaller scars
there may be less internal scarring when the procedures are performed in a minimally invasive fashion compared to standard open surgery.

Another similar procedure is called arthroscopy. Arthroscopic surgery is another minimally invasive surgical procedure in which an examination and sometimes treatment of damage of the interior of a joint is performed using an arthroscope that is inserted into the joint through a small incision. Arthroscopic procedures can be performed either to evaluate or to treat many orthopedic conditions including torn floating cartilage, torn surface cartilage, ACL reconstruction, and trimming damaged cartilage.

The advantage of arthroscopy over traditional open surgery is that the joint does not have to be opened up fully. Instead, only two small incisions are made—one for the arthroscope and one for the surgical instruments to be used in the knee cavity to fully remove the knee cap. This reduces recovery time and may increase the rate of surgical success due to fewer traumas to the connective tissue. It is especially useful for professional athletes, who frequently injure knee joints and require fast healing time. There is also less scarring, because of the smaller incisions. Irrigation fluid is used to distend the joint and make a surgical space. Sometimes these fluid leaks into the surrounding soft tissue causing extravasations and edema.

The key element in laparoscopic surgery is the use of a laparoscope. There are two types: (1) a telescopic rod lens system, that is usually connected to a video camera (single chip or three chip), or (2) a digital laparoscope where the charge-coupled device is placed at the end of the laparoscope, eliminating the rod lens system. Furthermore, attached is a fiber optic cable system connected to a ‘cold’ light source (halogen or xenon), to illuminate the operative field, inserted through a 5 mm or 10 mm cannula or trocar to view the operative field. The abdomen is usually insufflated, or essentially blown up like a balloon, with carbon dioxide gas. This elevates the abdominal wall above the internal organs like a dome to create a working and viewing space. CO₂ is used because it is common to the human body and can be absorbed by tissue and removed by the respiratory system. It is also non-flammable, which is important because electrosurgical devices are commonly used in laparoscopic procedures.

There are several instruments and devices for performing surgical endoscopic procedures including suction lines. For example, U.S. Pat. No. 7,083,601 describes a method for use in open and laparoscopic laser surgery and electrosurgery. The system includes a manual mode along with several automatic modes which can effectuate both suction and irrigation, either individually or simultaneously. In the various automatic modes, the suction and/or irrigation is automatically activated during activation of a medical apparatus for laser surgery or electrosurgery without requiring separate activation from the surgeon or operating room staff. Several safety features for monitoring the fluid control system are also incorporated within the system such as fluid sensors for detecting the absence of irrigation fluid, pressure sensors and vacuum systems for monitoring fluid pressure, fluid sensors for monitoring fluid volume, and warning signals for detecting empty containers. All the safety features are designed to automatically deactivate suction and/or irrigation means contained within the fluid control system upon detection of unsafe levels.

Another example is the U.S. Pat. No. 6,428,503 showing a surgical instrument for providing suction and/or irrigation mounted within the wall of an elongated housing or cannula such that the passageway through the housing is substantially unobstructed. A mounting notch is formed in the elongated housing and the surgical tool is formed with a mounting tab that matches the mounting notch. The mounting tab is positioned into the mounting notch and is substantially flush with the wall of the elongated housing. The mounting tab is then secured to the wall of the elongated housing, for example, by laser welding. The surgical tool can be formed with various tip shapes, including, but not limited to, a J hook, an L hook, a spatula, a needle, and a ball.

U.S. Pat. No. 5,685,877 describes a multiple tool laparoscopic surgical instrument that includes a hand piece, an outer conduit connected to the hand piece, four inner conduits within the outer conduit, an interior channel within the outer conduit, a hypodermic needle housed at least partially within a first one of the inner conduits, an electrocautery cutting tool housed at least partially within a second one of the inner conduit, and a needle electrode housed at least partially within a third one of the inner conduits. In operation, the interior channel of the outer conduit, which is connected to an external source of irrigating fluid and a suction device, is used to provide gentle irrigation as well as suction. A fourth one of the inner conduits, which preferably has a cross sectional area smaller than that of the interior channel of the outer conduit, is also connected to an external source of irrigating fluid to provide more vigorous irrigation. The hypodermic needle, electrocautery cutting tool and the needle electrode are individually and selectively extended and retracted to perform desired surgical tasks with each such tool.

Furthermore, U.S. Pat. No. 5,569,164 is a surgical laparoscopic apparatus comprising a protector tube and a grip mounted onto the rear end thereof. At least one rod-shaped instrument is arranged in an axially removable way in an axial guide channel provided in the protector tube and the handgrip. A dual purpose flushing/suction channel extends parallel thereto in the protector tube and partially also in the handgrip and passes through the handgrip to external flush and suction connectors. A twin-position three-way valve is arranged in the handgrip adjacent to the guide channel and, in the one position, connects the flushing/suction channel to the flush and suction connectors and, in the other position, connects the guide channel to the flush connector or suction connector.

Even though the above cited methods address some of the needs of the market, a more effective suction device capable of providing a blood removal effect on the surgical site while the surgical procedure is being performed is still desired.

### SUMMARY OF THE INVENTION

This invention is directed to a novel suction device especially designed for laparoscopic and/or arthroscopic instruments, including at least two cylindrical suction ports located at both sides of the distal end of the laparoscopic and/or arthroscopic instrument, each having a sharp traversing blade.

In one general aspect of the present invention, the suction device comprises two suction ports connected to respective suction lines in turn connected to a suction pump for delivering blood, coagula and debris to a disposal recipient. Connected to said suction line.

Another aspect of the present invention provides a suction device comprising four suction ports surrounding the distal end of the laparoscopic and/or arthroscopic instrument, each of which having a traverse sharp edge and connected to a suction line.

Some of the advantage of the present invention may be summarized as:

It creates an even suction effect on the surgical field;

Allows the removal not only of blood but also solid debris and coagula without risk of blocking the suction line;

Allows the destruction of any coagula or debris by cutting them with the sharp blade incorporated into the suction port;

Avoids the cumbersome procedure of taking the instruments out of the patient for inserting the suction device for removing blood and coagula from the surgical field.

It may be used in laparoscopic robotic surgery procedures, saving surgical time, needing less anesthesia, and less complications.

In summary, the present invention is related to a suction device especially designed for laparoscopic and/or arthroscopic instruments, including at least two cylindrical suction ports located at both sides of the distal end of the laparoscopic and/or arthroscopic instrument. Each suction port has inside and next to the distal end thereof at least one sharp traversing blade. Each suction port is connected to a suction line in turn connected to a suction pump. The external end of said suction line is connected to a disposal recipient into which the debris, blood, coagula, etc. removed from the surgical site will be disposed.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is a general schematic perspective view of a laparoscopic and/or arthroscopic biopsy forceps including the suction device in accordance with the present invention. It shows the moment in which the tip of the instruments approaches the tissue to be treated.

FIG. 2 is another perspective view of the laparoscopic and/or arthroscopic instrument showing the moment in which the forceps is open by moving backwards the mechanical command. In this figure it could also be seen the suction line coming from the central lumen of the instrument.

FIG. 3 is another perspective view showing the moment in which the forceps is open and is approaching a tissue.

FIG. 4 is another perspective view showing how the forceps command is moving forward closing the forceps jaws.

FIG. 5 is another perspective view showing the forceps grabbing the tissue, blood flowing from the tissue and being sucked by the suction ports and then blood and debris going through the suction line.

FIG. 6 is similar to FIG. 5 but this time illustrating blood going through the suction line towards the disposal bag.

FIG. 7 is a frontal perspective view showing the forceps grabbing the stomach tissue, and blood being removed from the surgical site by the suction ports and then going through the suction line.

FIG. 8 shows schematically the sucked blood going through the suction line.

FIG. 9 shows schematically the sucked blood going through the suction line and entering into the disposal recipient.

FIG. 10 is a perspective view of a second embodiment in which the laparoscopic and/or arthroscopic instrument is now a laparoscopic and/or arthroscopic scissor. It shows the moment in which the scissor is open by moving backwards the mechanical command. In this figure it could also be seen the suction line coming from the central lumen of the instrument.

FIG. 11 is another perspective view showing the moment in which the laparoscopic and/or arthroscopic scissor is open and is approaching a stomach tissue.

FIG. 12 is a frontal perspective view showing the scissor cutting the stomach tissue, and blood flowing and being removed from the surgical site by the suction ports and then going through the suction line, finally:

FIG. 13 shows schematically the sucked blood going through the suction line and entering into the disposal recipient.

### DETAILED DESCRIPTION OF REPRESENTATIVE EMBODIMENTS

Shown throughout the Figures, the invention is directed to a suction device 10 for laparoscopic and/or arthroscopic instruments 100, including at least two cylindrical suction ports 11 located at both sides of the distal end 101 of the laparoscopic and/or arthroscopic instrument 100.

Each suction port 11 is defined by a cylindrical tube located one at each side of the distal end 101 of the laparoscopic and/or arthroscopic instrument 100. Each tube 11 has inside and next to the distal end thereof at least one sharp traversing blade 12. This blade has the very important mission of cutting any debris or coagulum the suction port may remove from the surgical site.

Also each suction port 11 is connected to a suction line 13 in turn connected to a suction pump (not illustrated). The external end 14 of said suction line 13 is connected to a disposal recipient 120 into which debris, blood, coagula, etc. removed from the surgical site will be disposed.

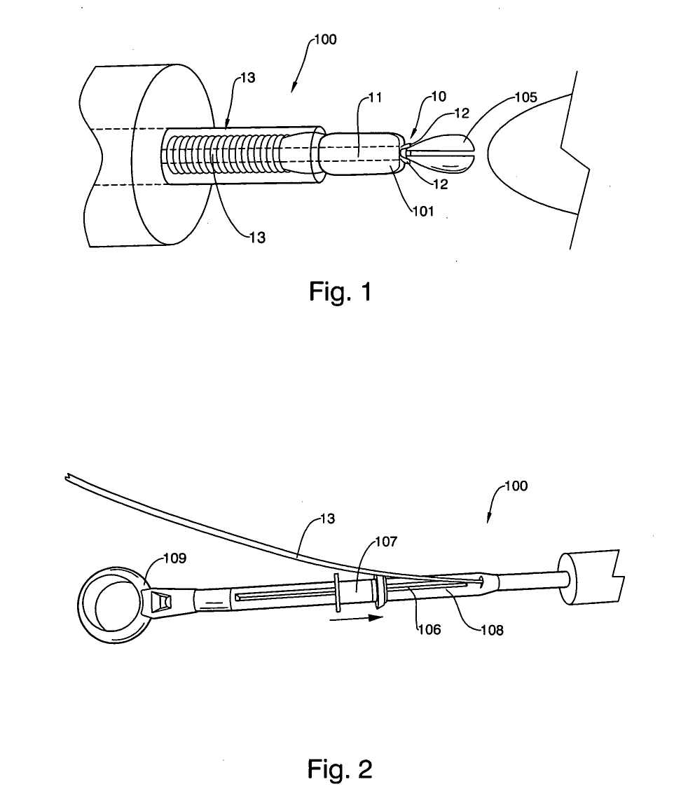

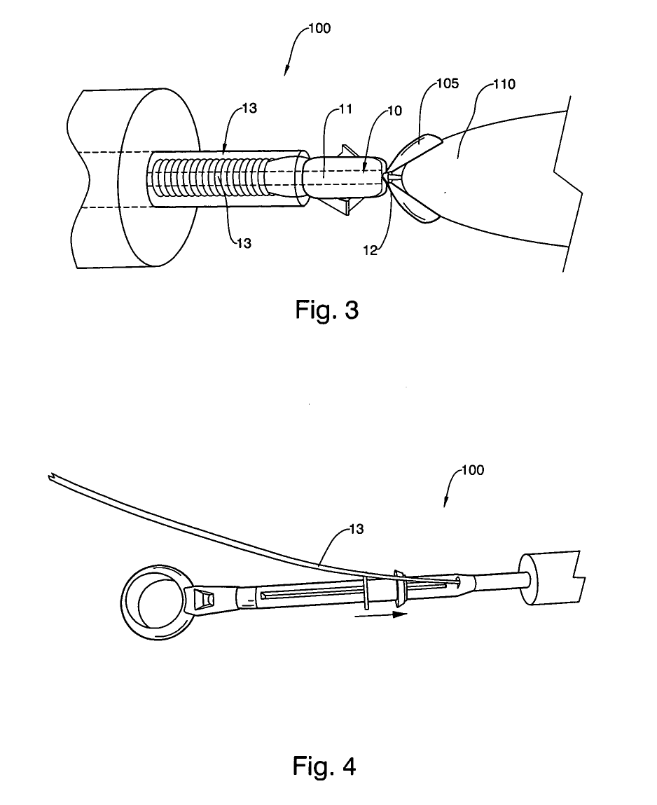

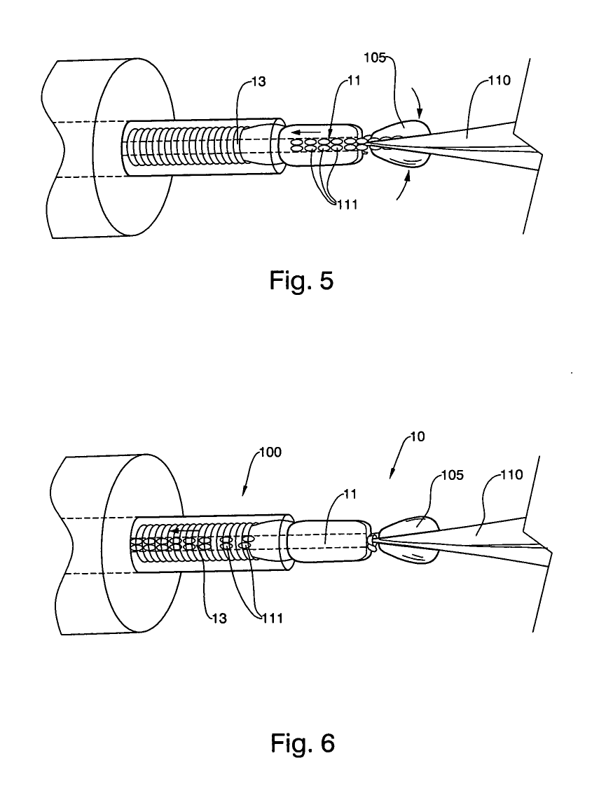

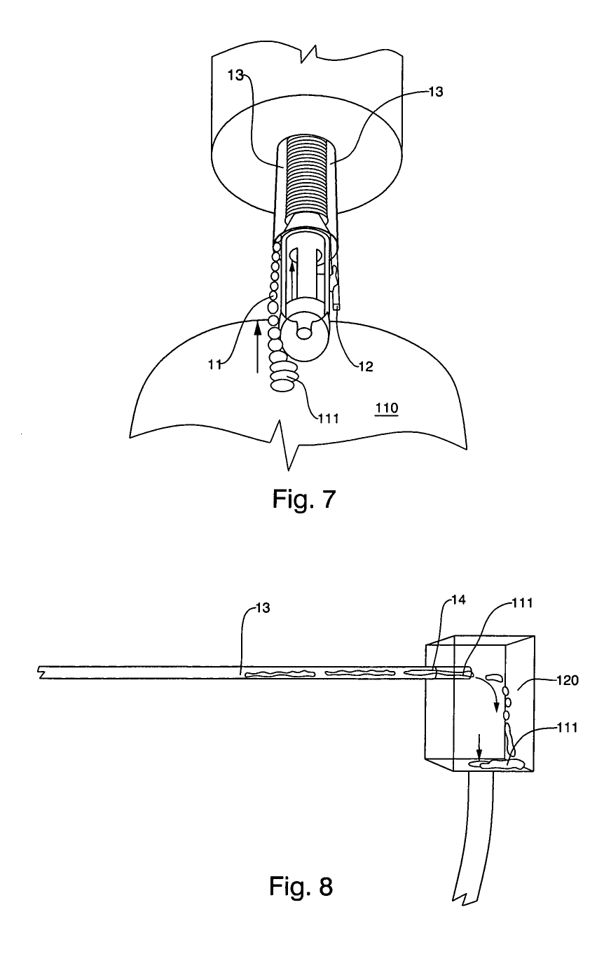

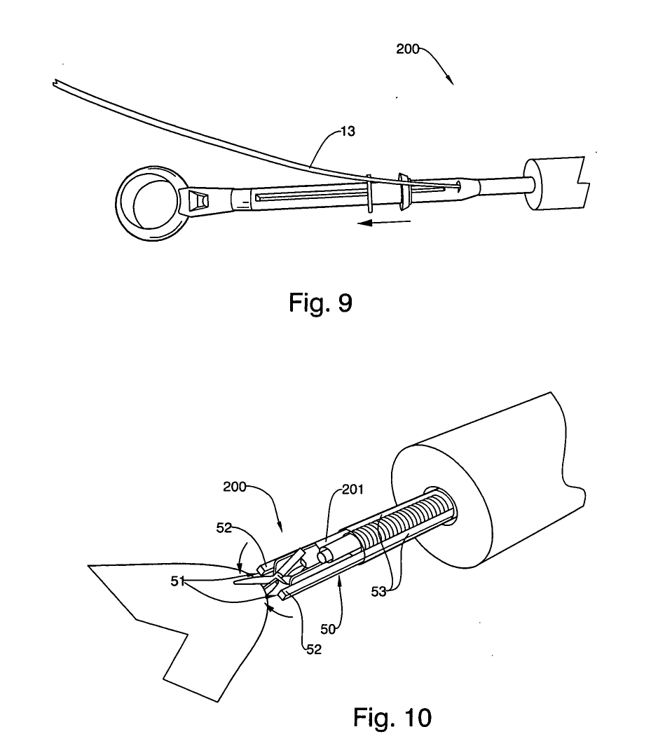

FIGS. 1-9 illustrate a first embodiment of the present invention in which the laparoscopic and/or arthroscopic instrument 100 is a laparoscopic and/or arthroscopic biopsy forceps. As usual, said instrument includes at its distal end two movable grabbing jaws 105 pivotally connected to the distal end 101. Said jaws are connected through a commanding cable 106 to a control device 107. In this embodiment this control device comprises a mechanical piston linearly movable on longitudinal tracks 108. A handle 109 at the external end allows the surgeon to manipulate it easily.

When the above mentioned piston 107 is moved backwards (FIG. 2) said cable 108 is pulled and jaws 105 are open. When said piston is moved forward, jaws 105 are closed. This mechanism is not disclosed in detail as it is a very well known mechanism and it is not part of the present invention.

The laparoscopic and/or arthroscopic procedure starts when instrument 100, such as a laparoscopic and/or arthroscopic biopsy forceps, is introduced into the patient's body through a trocar or a small incision in the abdominal area of the patient's body. The control command 107 is moved backwards to open the forceps jaws 105. Then forceps 100 is approached to the tissue section 110 to be studied. The suction pump (not illustrated) is activated so as to create a suction effect of both suction ports 11. Thus, a suction effect is created around the forceps grabbing the stomach tissue 110. Blood 111 is removed through the suction ports 11 while the forceps grabs tissue 110. Blood 111, coagula and debris sucked by the suction port 11 from the surgical site are then transferred by this suction effect to the suction line 13 towards a disposal bag 120.

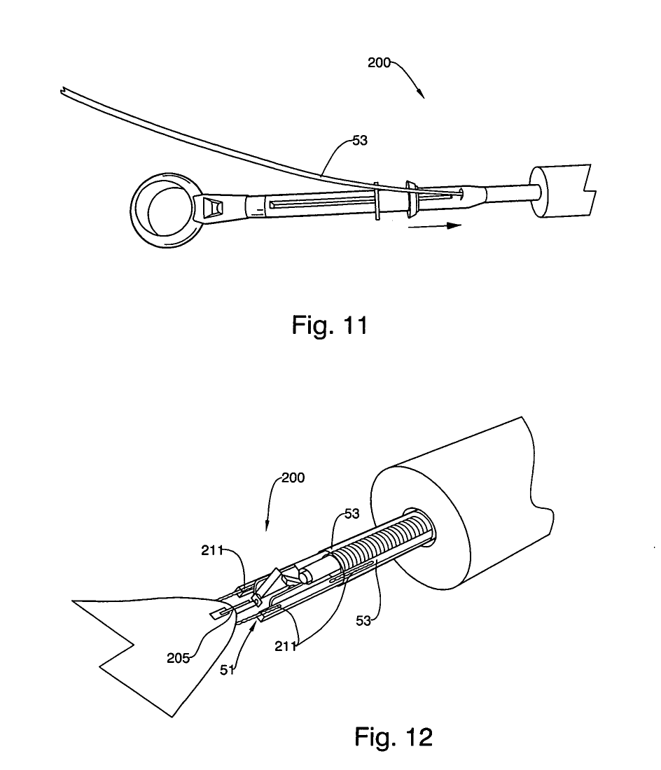

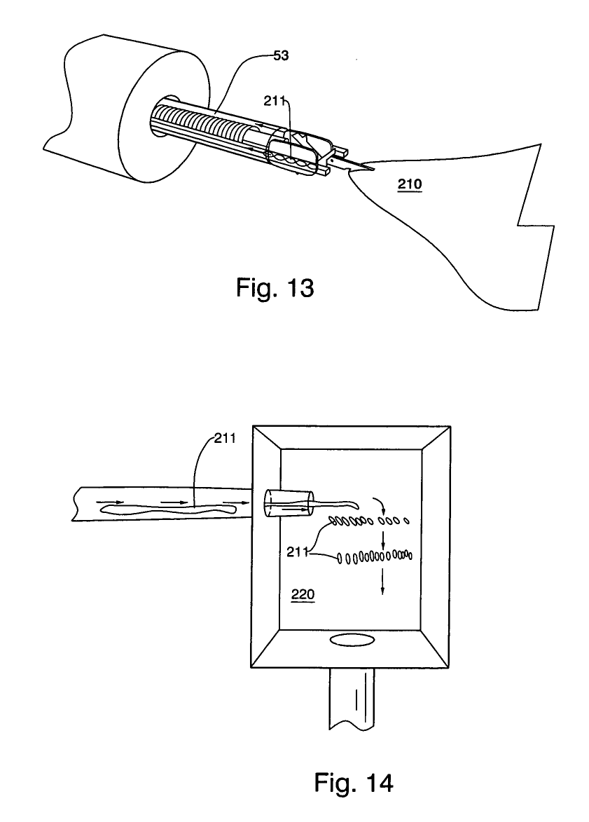

FIGS. 10-13 illustrates a second embodiment of the present invention, comprising this time a suction device 50 for a laparoscopic and/or arthroscopic scissor 200, including at least two cylindrical suction ports 51 located at both sides of the distal end 201 of the laparoscopic and/or arthroscopic scissor 200.

Each suction port 51 is defined by a cylindrical tube located one at each side of the distal end 201 of the laparoscopic and/or arthroscopic instrument 200. Each tube 51 has inside and next to the distal end thereof at least one sharp traversing blade 52. This blade has the very important mission of cutting any debris or coagulum the suction port may remove from the surgical site.

Also each suction port 51 is connected to a suction line 53 in turn connected to a suction pump (not illustrated). The external end 54 of said suction line 53 is connected to a disposal recipient 220 into which debris, blood, coagula, etc. removed from the surgical site will be disposed.

As usual, said laparoscopic and/or arthroscopic scissor includes at its distal end two movable blades 205 pivotally connected to the distal end 201. Said blades are connected through a commanding cable 206 to a control device 207. In this embodiment this control device 207 comprises a mechanical piston linearly movable on longitudinal tracks 208. A handle 209 at the external end allows the surgeon to manipulate it easily.

When the above mentioned piston 207 is moved backwards said cable 208 is pulled and blades 205 are open. When said piston is moved forward (FIG. 12), blades 205 are closed and the tissue is cut. This mechanism is not disclosed in detail as is not part of the present invention.

The laparoscopic and/or arthroscopic procedure starts when instrument 200, such as a laparoscopic and/or arthroscopic scissor, is introduced into the patient's body through small incisions, for example, in the abdominal area. The control command 207 is moved backwards to open the scissor blades 205. Then scissor 200 is approached to the tissue section 210 to be studied. The suction pump (not illustrated) is activated to create a suction effect of both suction ports 51. Thus, a suction effect is created around the scissor blades cutting the stomach tissue 210. Blood 211 is removed through suction ports 51 while the scissor cuts tissue 210. Blood 211 and/or coagula and/or debris sucked by the suction port 51 from the surgical site are then transferred by this suction effect to the suction line 53 towards a disposal bag 220.

While the preferred embodiments of the invention have been described above, it will be recognized and understood that various modifications can be made in the invention and the appended claims are intended to cover all such modifications which may fall within the spirit and scope of the invention

I claim:

1. Suction device for laparoscopic and/or arthroscopic instruments, including at least two cylindrical suction ports located at both sides of the distal end of the laparoscopic and/or arthroscopic instrument; each suction port has inside and next to the distal end thereof at least one sharp traversing blade; and each suction port is connected to a suction line in turn connected to a suction pump; the external end of said suction line is connected to a disposal recipient into which the debris, blood, coagula etc removed from the surgical site will be disposed.

2. The suction device in accordance with claim 1, wherein the device has four suction ports surrounding the distal end of the laparoscopic and/or arthroscopic instrument to which it is installed.

3. The suction device in accordance with claim 1, wherein inside each suction port a diametrically disposed sharp blade defines a cutting means inside said port for cutting any debris, coagula or the like removed from the surgical field during the laparoscopic and/or arthroscopic procedure.

4. The suction device in accordance with claim 1, wherein said ports are connected to two different parallel suction tubes which are part of the suction line.

5. The suction device in accordance with claim 1, wherein the suction lines are connected to a disposal bag especially design for containing and disposing biological material.

6. A suction method for laparoscopic and/or arthroscopic procedures, comprising the steps of:
a) Introducing the laparoscopic and/or arthroscopic instrument into the patient's body through a small incision;
b) Activating the control command located at the instrument external end to open the instrument;
c) Approaching the instrument to the stomach section to be studied;
d) Activating the suction pump;
e) Creating a suction effect around the instrument grabbing the stomach tissue;
f) Removing blood through the suction ports while the forceps grabs the tissue;
g) Sucking blood, coagula and debris by the suction port from the surgical site;
h) Transferring said blood, coagula and debris by this suction effect to the suction line towards a disposal bag.

7. The method of claim 6, wherein the instrument is a laparoscopic and/or arthroscopic biopsy forceps
